(19) United States
(12) Patent Application Publication
(10) Pub. No.: US 2023/0277740 A1
(43) Pub. Date: Sep. 7, 2023

(54) METHOD AND APPARATUS FOR MEASUREMENT OF AN ULTRAFILTRATION RATE IN A RENAL REPLACEMENT THERAPY DEVICE

(71) Applicant: Transonic systems Inc., Ithaca, NY (US)

(72) Inventors: Nikolai M. Krivitski, Ithaca, NY (US); Fahimeh Salehpour, San Jose, CA (US)

(21) Appl. No.: 17/684,012

(22) Filed: Mar. 1, 2022

(51) Int. Cl. A61M 1/34

(52) U.S. Cl. CPC A61M 1/3403; A61M 2205/3334

(57) ABSTRACT

A system of identifying an ultrafiltration rate in a renal replacement therapy device is provided, wherein the system includes a controller connected to a first flow sensor obtaining flow rate data from a blood withdrawal line and a second sensor obtaining flow rate data from a blood delivery line. The controller calibrates, such as matches, the first flow sensor and the second flow sensor from flow measurements during periods of known ultrafiltration by the renal replacement therapy device. The controller is further configured to perform periodic equalization of the flow sensors, at a known ultrafiltration rate during the treatment session. The controller can employ flow rate data from the calibrated or equalized first flow sensor and the second flow sensor to calculate an ultrafiltration rate of the renal replacement therapy device based on the measured blood flow into and out of the renal replacement therapy device. The calibration can be performed before, during, or after blood treatment in a treatment session.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO A SEQUENCE LISTING

Not applicable.

### STATEMENT REGARDING PRIOR DISCLOSURES BY THE INVENTOR OR A JOINT INVENTOR

Not applicable.

### BACKGROUND OF THE INVENTION

### Field of the Invention

The present disclosure relates to an apparatus and method for identifying, with improved accuracy, an ultrafiltration rate of a renal replacement therapy device based on a measured blood flow in a blood withdrawal line to the renal replacement therapy device and a measured blood flow in a blood delivery line from the renal replacement therapy device.

### Description of Related Art

A variety of different medical treatments relate to the delivery of fluid to, through and/or from a patient, such as the delivery of blood between a patient and an extracorporeal system. For example, hemodialysis, hemofiltration, and hemodiafiltration are all renal replacement therapies that remove waste, toxins, and excess water from the blood, wherein during these treatments, the patient is connected to an extracorporeal system having a treatment or renal replacement device, and the blood is pumped through the system and the device, wherein the waste, toxins, and fluid are removed from the blood, and the cleaned blood is returned to the patient.

In dialysis therapy, blood is extracted from the body and conveyed through the extracorporeal system to a dialyzer. Blood flows into the dialyzer via a blood inflow port, flows through hollow fiber membranes and out from an outflow port, and is returned to the patient. Simultaneously, dialysate is supplied into the casing via a dialysate inflow/outflow port to fill between the hollow fiber membranes. The blood and dialysate undergo substance exchange via the hollow fiber membranes.

In order to substitute for the function of the kidney in adjusting the water content, the dialyzer is used to perform fluid removal for discharging excessive water content out of the body during the dialysis therapy. In the fluid removal process, the flow of dialysate is controlled to increase the quantity of outflow of dialysate compared to the quantity of inflow of dialysate, so that negative pressure is generated within the casing thereby extracting the water content within the blood across the semi-permeable membrane toward the dialysate (i.e., by ultrafiltration).

The withdrawal of fluid in the dialyzer, also known as ultrafiltration, is given by the difference between the spent dialysate pumped out of the dialyzer and the fresh dialysate pumped into the dialyzer. Because of the large volume of dialysate that is exposed to the membrane in the dialyzer during dialysis therapy, there is a need for accurate control of the ultrafiltration. In hemodialysis for example, typically about 200 liters of dialysate are passed through the dialyzer during a treatment session. The target amount of ultrafiltrate during a treatment session is typically about 2 to 3 liters and may need to be controlled with a maximum deviation of the order of only 0.1 to 0.2 liter. Accordingly, in this example, ultrafiltration may need to be controlled with a maximum error of approximately 1:1000 in relation to the total flow of dialysate.

Currently, the standard of accurate ultrafiltration measurements requires expensive equipment to be deployed in hemodialysis machines. For example, current systems include two scale set before and after the dialyzer on dialysate side. However, these systems are relatively complicated and can impart operator error.

### BRIEF SUMMARY OF THE INVENTION

The present disclosure addresses a current issue in blood flow measurement systems with flow sensors for measuring ultrafiltration, where the errors in measurement from the flow sensors are much greater than the necessary accuracy of measurements for determining fluid removal by a renal replacement therapy device.

For example, a current specification of an error “d” in blood flow measurement in hemodialysis lines by manufacturers of ultrasound transit time sensors is d = ± 6%. This error is caused by multiple factors including: error in factory calibration of the equipment, nonlinearity of the flow vs. recorded voltage, the influence of blood temperature and ambient temperature on the functioning of the sensors and their electronic performance, deviation of the tubing in the system from a factory calibration, deviations within the tubing used in the field, as well as variations in the density of the blood, including hematocrit and ion concentration. These flow measurement errors can manifest as: (i) deviation of the slope (angle) of a curve or graph of recorded flow vs pump flow setting (or actual flow), and (ii) fluctuation of the Y-intercept (FIGS. 3A and 3B).

This means that for an error d = ± 6%, in an exemplary blood flow of 300 ml/min, the error will be within ± 18 ml/min. Considering that ultrafiltration is the difference between Q_{A}-Q_{V}, then the error, if independent from variables, will be [(da)2+(dv)2]½ ≈ ± 8.5% or ≈ ± 25 ml/min at 300 ml/min flow rate in the renal replacement therapy device. Considering for example, the removal of fluid from a patient through an ultrafiltration rate (UF) is typically on the order of 600 ml/hour which is 10 ml/min. Therefore, if one wants to measure UF with the error within 10% of the flow rate, this requires UF needs to be measured with error ± 1 ml/min (for UF = 10 ml/min). Thus, an error of error ± 1 ml/min for a 300 ml/min blood flow through the renal replacement therapy device requires a measurement within ± 0.3 % error. The available error of UF measured by current flow sensors is approximately ± 25 ml/min which is almost 30 times larger than the error that is needed for providing useful UF measurements.

Generally, the present method and apparatus encompass measuring, with calibrated flow sensors, blood flow of an input line and an output line of the renal replacement therapy device, and based on a difference between blood flow into the renal replacement therapy device (blood flow of the input line) and blood flow out of the renal replacement therapy device (blood flow out of the output line), and assessing fluild removal (ultrafiltration) during a blood treatment session, wherein the ultrafiltration can be measured substantially continuously during the treatment session.

The present disclosure contemplates calibrating a first flow sensor measuring blood flow of the input line and a second flow sensor measuring blood flow out of the output line, wherein the difference in measured flow is used to determine ultrafiltration. It is understood calibrated flow sensors includes the first flow sensor being calibrated, or the second flow sensor being calibrated, or both the first flow sensor and the second flow sensor being calibrated.

In general, the disclosure contemplates a calibration of the flow sensors, wherein the calibration can be a flow sensor matching or a flow sensor equalization. In the flow sensor matching, the flow sensors are exposed to a plurality of different common flow rates through the extracorporeal circuit, with a known, such as zero ultrafiltration, wherein the flow sensors are standardized to provide the same measure of the same flow. That is, the matching provides that measurement signals from the flow sensors as disposed within an extracorporeal circuit will provide equal readings of a common flow. In the flow sensor equalization, the matched flow sensors are subsequently exposed to a common flow, with a known ultrafiltration rate, such as a zero ultrafiltration rate, and the flow sensors are equalized to provide the same measure for the common flow, by either adjusting the first flow sensor, the second flow sensor or both flow sensors so as to provide an equal measure of the common flow. That is, the equalization provides that the signals from one flow sensor are equalized to the signals from the second flow sensor. It is contemplated that equalization can be performed periodically during a treatment session.

The present disclosure provides an apparatus for measuring ultrafiltration and particularly an ultrafiltration rate of a renal replacement therapy device in an extracorporeal circuit, wherein the renal replacement therapy device includes or is operably coupled to a blood input line delivering blood to the renal replacement therapy device, a pump, a permeable membrane, a blood output line passing blood from the renal replacement therapy device, and is configured to establish a known, such as zero ultrafiltration rate and a target ultrafiltration rate, the apparatus including a first flow sensor configured to measure a blood input line flow in the blood input line; a second flow sensor configured to measure a blood output line flow in the blood output line; and a controller connected to the first flow sensor and the second flow sensor, wherein the controller is configured to calibrate, based on blood flow in the extracorporeal circuit, the first flow sensor and the second flow sensor. It is contemplated the controller is further configured to determine an ultrafiltration rate and ultrafiltration volume of the renal replacement therapy device.

It is understood the calibration of the first flow sensor and the second flow sensor can include matching the flow sensors such that the flow sensors indicate an equal measure of flow at a given pump flow rate and a known, such as zero ultrafiltration, and particularly with the specific tubing and environmental conditions of the extracorporeal circuit and the treatment session. That is, the calibration is not a bench or factory calibration, but rather can be a matching of the flow sensors for accommodating the particular equipment of the given extracorporeal circuit and treatment session, wherein the first flow sensor and the second flow sensor are matched to each other based on blood flow through the extracorporeal circuit. The matching encompasses an adjustment of the flow sensor or the obtained measurements which account for external factors or to allow comparison with the measurement data from another flow sensor in the system. As set forth below, the matching can include adjusting the measurements of one or both of the now sensors, employing a compensating or adjusting factor to the measurements, or signals, of one or both of the flow sensors, as well as a lookup table, or a mechanical adjustment of the respective flow sensor. Thus, it can be described that the matched first flow sensor and the second flow sensor are calibrated.

The calibration can also include equalizing the first flow sensor and the second flow sensor or removing any offset of the signals at a common flow rate at a known or zero ultrafiltration. That is, it is understood the calibration does not require the signals generated by the respective flow sensor be equal, but rather the signal from one flow sensor is equalized to the signal from the remaining flow sensor.

Calibrating the first flow sensor to the second flow sensor means calibrating the sensors such that the signal from each sensor at a common flow rate and a known or zero ultrafiltration are taken or treated as indicating the same flow rate. Matching the first flow sensor to the second flow sensor for at least two common flow rates at a known or zero ultrafiltration in the extracorporeal circuit provides a two point calibration. Equalizing the first flow sensor and the second flow sensor at a single common flow rate and a known or zero ultrafiltration are taken or treated as indicating the same flow rate provides a single point calibration. That is, the present disclosure contemplates one point calibration, as well as two point calibration to re-scale the output of the respective flow sensor. Two point calibration can be used in cases where the sensor output is known to be sufficiently linear over the measurement range and is capable of correcting both slope and offset errors.

It is understood that zero ultrafiltration rate means no liquid is passing through the permeable membrane in the renal replacement therapy device. The zero ultrafiltration rate can be obtained by, but is not limited to, stopping ultrafiltration or bypassing the renal replacement therapy device to provide an absolute zero ultrafiltration rate, It is further contemplated that a known ultrafiltration rate instead of a zero ultrafiltration rate or in combination with zero ultrafiltration rate can be implemented to be used in the present system for the calibration process. For purposes of description, the term known ultrafiltration rate will be used, wherein a zero ultrafiltration rate is an exemplary known ultrafiltration rate.

The present method and apparatus can improve the accuracy in measuring ultrafiltration in the renal replacement treatment session by the following steps: (i) before starting ultrafiltration in the treatment session, calibrating, such as matching, a first flow sensor and a second flow sensor against each other for at least two flow rates, or through a given flow range; (ii) during the treatment session, periodically calibrating, such as equalizing, the first flow sensor and the second flow sensor against each other at a known ultrafiltration rate to remove any offset between the flow sensors occurring or generated during the renal replacement treatment session; (iii) averaging flow measurements of the first flow sensor and the second flow sensor over a sufficient period of time to reduce, or substantially eliminate, flow measurement error caused by pulsations in the measured flow; (iv) assessing a target ultrafiltration rate based on measurements from the calibrated first flow sensor and the second flow sensor (such as a difference between adjusted flow measurements from the first flow sensor and the second flow sensor), and (v) estimating an amount of fluid removal in the renal replacement treatment session. It is contemplated that calibrating the first flow sensor and the second flow sensor includes matching the sensors, which can remove potential sensor factory calibration inaccuracy.

The present disclosure provides a method of matching a first flow sensor and a second flow sensor to compensate for the real-time conditions such as tubing material, temperature, etc. of the treatment session and can be done before, during or after treatment of the blood in the treatment session. The matching includes the steps of (i) registering, at a known or zero ultrafiltration rate, a flow measured by the first flow sensor and the second flow sensor, at a minimum of two pump flow rates (within an expected flow range during the treatment session); (ii) identifying a slope of a curve of measured flow for the first flow sensor and the second flow sensor; (iii) identifying a correction factor to match a slope of the first flow sensor and the second flow sensor; and (iv) applying the correction factor to a flow measured during ultrafiltration in the treatment session by at least one of the first flow sensor and the second flow sensor. It is understood that matching can be done before, during or after flow measurement from the flow sensors during a treatment session, wherein the measurements are subsequently adjusted to accommodate the matching of the flow sensors.

Matching the flow sensors can be performed at any time during the treatment session; however, it is advantageous to identify the calibration or correction factor prior to the treatment session. In case of calibrating the flow sensors during or at the end of the treatment session, the ultrafiltration rate or amount of fluid removed is (re)calculated upon application of the sensor calibration.

The present disclosure also provides a method of calibrating the flow sensors, such as equalizing the first flow sensor and the second flow sensor during the treatment session to remove potential offset between the sensors, that may occur during the treatment session. The method includes the steps of (i) turning off ultrafiltration (operating at a known or zero ultrafiltration rate) for a short period of time (e.g between 1 second and 360 seconds, or between 10 seconds and 90 seconds, or on the order 30 seconds +/- 15 seconds); (ii) identifying an offset between the first and the second flow sensor by calculating difference between the respective flow measured by the sensors; and (iii) calibrating, by equalizing, subsequent flow measurements of the first and second flow sensors by the identified offset.

It is recognized that the blood flow signals registered by the first and the second flow sensors are highly pulsatile which can produce errors in an instantaneous flow measurement (FIG. 5). The present method and apparatus provide a method to reduce error in flow measurement due to pulsatile flow signal. The method contemplates a sufficient period of data collection for the flow measurement to include at least one full cycle of pulsatile flow signal within the averaging to reduce, or eliminate, flow measurement error from the fluctuation in pulsations. Measuring the flow over a sufficient time reduces the error from the presence the pulsations in the flow and averaging over a period reduces the error from fluctuations in the pulsations. It is further contemplated the flow measurements can be taken at a common point within the pulsatile cycle.

In one configuration, the present disclosure provides an apparatus for measuring an ultrafiltration rate of a renal replacement therapy device in an extracorporeal circuit having a pump, the renal replacement therapy device configured to establish a first and a second known ultrafiltration rate, which can be a zero ultrafiltration rate, and a target ultrafiltration rate, the renal replacement therapy device having a blood input line delivering blood to the renal replacement therapy device, a permeable membrane, a blood output line passing blood from the renal replacement therapy device, the apparatus including a first flow sensor configured to measure a flow rate in the blood input line; a second flow sensor configured to measure a flow rate in the blood output line; and a controller connected to the first flow sensor and the second flow sensor, the controller configured to (i) register a first flow rate measured by the first flow sensor and a first flow rate measured by the second flow sensor each at a first pump rate and the first known ultrafiltration rate, (ii) register a second flow rate measured by the first flow sensor and a second flow rate measured by the second flow sensor each at a second pump rate and the second known ultrafiltration rate, (iii) calibrate at least one of the first flow sensor and the second flow sensor corresponding to the first flow rate measured by the first flow sensor, the first flow rate measured by the second flow sensor, the second flow rate measured by the first flow sensor, and the second flow rate measured by the second flow sensor, and (iv) identify the target ultrafiltration rate from a third flow rate measured by the first flow sensor and a third flow rate measured by the second flow sensor during the target ultrafiltration rate,

A method is provided of measuring a target ultrafiltration rate by a renal replacement therapy device in an extracorporeal circuit having a pump, the renal replacement therapy device configured to provide a first known ultrafiltration rate, a second known ultrafiltration rate, and the target ultrafiltration rate, and having a blood input line delivering blood to the renal replacement therapy device, a permeable membrane, and a blood output line passing blood from the renal replacement therapy device, the method including measuring, at a first pump flow rate and the first known ultrafiltration rate, (i) a first flow rate in the blood input line by a first flow sensor and (ii) a first flow rate in the blood output line by a second flow sensor; measuring, at a second pump flow rate and the second known ultrafiltration rate, (i) a second flow rate in the blood input line by the first flow sensor and (ii) a second flow rate in the blood output line by the second flow sensor; calibrating at least one of the first flow sensor and the second flow sensor corresponding to the first flow rate in the blood input line measured by the first flow sensor, the first flow rate in the blood output line measured by the second flow sensor, the second flow rate in the blood input line measured by the first flow sensor, and the second flow rate in the blood output line measured by the second flow sensor; measuring, during the target ultrafiltration rate and a target pump flow rate, (i) a third blood flow rate in the blood input line by the calibrated first flow sensor and (ii) a third blood flow rate in the blood output line flow by the calibrated first flow sensor; and identifying, at the target ultrafiltration rate and the target pump flow rate, the target ultrafiltration rate corresponding to the third blood flow rate in the blood input line measured by the calibrated first flow sensor and the third blood flow rate in the blood output line flow measured by the calibrated second flow sensor.

A further method is provided in the present disclosure of measuring a target ultrafiltration rate by a renal replacement therapy device in an extracorporeal circuit having a pump, the renal replacement therapy device configured to provide a first known, such as zero, ultrafiltration rate, a second known, such as zero, ultrafiltration rate, and the target ultrafiltration rate, and having a blood input line delivering blood to the renal replacement therapy device, a permeable membrane, and a blood output line passing blood from the renal replacement therapy device, the method including calibrating a first flow sensor configured to sense a flow rate in the blood input line and a second flow sensor configured to sense a flow rate in the blood output line, wherein the calibrating corresponds to at least (i) a first flow rate in the extracorporeal circuit measured by each of the first flow sensor and the second flow sensor at a first pump flow rate and the first known ultrafiltration rate, and (ii) a second flow rate in the extracorporeal circuit measured by each of the first flow sensor and the second flow sensor at a second pump flow rate and the second known ultrafiltration rate; and identifying the target ultrafiltration rate corresponding to a third flow rate measured by the calibrated first flow sensor and the second flow sensor at a given pump flow rate and the target ultrafiltration rate.

The following will describe embodiments of the present disclosure, but it should be appreciated that the present disclosure is not limited to the described embodiments and various modifications of the invention are possible without departing from the basic principles. The scope of the present disclosure is therefore to be determined solely by the appended claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

FIG. 1 is a schematic of a representative extracorporeal circuit including a renal replacement therapy device.

FIG. 2 is a graph of a flow profile through the extracorporeal circuit during a treatment session showing calibration including flow sensor matching and flow sensor equalization,

FIG. 3A and FIG. 3B are comparison graphs of a flow sensor matching procedure, where the curves on the graph of FIG. 3A show the flow measurement of the first flow sensor and the second flow (venous) sensor for multiple pump flow rate settings at a zero ultrafiltration rate. The curves in the graph of FIG. 3B show the flow rate data from the second flow (venous) sensor is calibrated, such as by adjusting the flow rate data from the first flow (arterial) sensor.

FIG. 4A and FIG. 4B are graphs showing a flow sensor equalizing procedure, wherein in every d period, the ultrafiltration UF of the renal replacement therapy device is turned off (UF = 0) for calibrating, such as matching, equalizing or adjusting the first (arterial) flow sensor arterial and the second (venous) sensor against each other. The graph in FIG. 4A shows the flow measurement before calibrating, such as equalizing the flow sensors periodically, and the graph in FIG. 4B shows flow measurement after applying periodic calibration.

FIGS. 5A - 5G are a series of graphs showing the impact of the duration of flow measurements that are averaged to obtain a flow measurement (shown in a shaded panel in each graph). From FIG. 5A to FIG. 5G, the time period (shown as shaded) is increased to include more cycles in the averaging. FIGS. 5A - 5G show the effect of the averaging time period on reducing the impact of the pulsatile component on the measured flow.

FIG. 6 is a representative flow chart of calibrating the flow sensors by matching the flow sensors in the extracorporeal circuit.

FIG. 7 is a representative flow chart of calibrating the flow sensors by equalizing the flow sensors in the extracorporeal circuit.

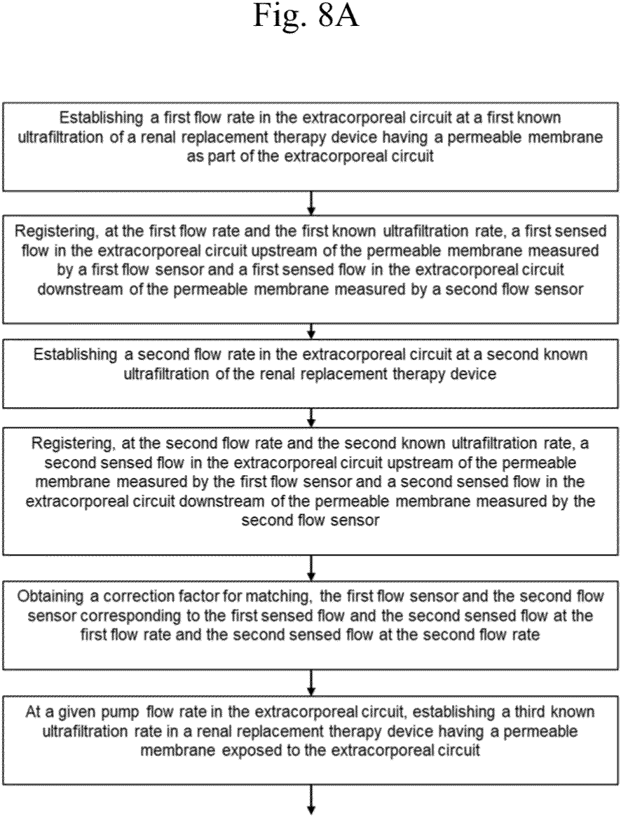

FIGS. 8A and 8B are representative flow charts of calibrating the flow sensors by matching and equalizing the flow sensors in the extracorporeal circuit.

### DETAILED DESCRIPTION OF THE INVENTION

Generally, the present disclosure is directed to a renal replacement therapy device 130. In one configuration, the present disclosure is directed to an extracorporeal renal replacement therapy device 130, capable of generating a pressure differential across a semi-permeable membrane to provide ultrafiltration and hence an ultrafiltration rate.

Renal replacement therapy is directed to two primary objectives, the first objective is to remove kidney failure-related toxins and the second objective is to remove excess water and salt from the blood. Generally, renal replacement therapy employs two physiologies for solute and fluid movement. Both methods require sequestration of blood on one side of a semi-permeable membrane.

These treatments may be performed by pumping a dialysis fluid through a treatment device such as the renal replacement therapy device 130, commonly referred to as a dialyzer, in which fluid and substances are transported over a semi-permeable membrane. Diffusive mass transport through the membrane is predominant in hemodialysis (HD), whereas hemofiltration (HF) uses mainly convective mass transport through the semi-permeable membrane; and hemodiafiltration (HDF) is a combination of the two methods.

In diffusive clearance (dialysis), solute moves down its concentration gradient, from areas of higher concentration to areas of lower concentration, The solute must be of appropriate size and charge to pass through a semi-permeable membrane. By passing fluid across the membrane countercurrent to blood flow, equilibration of plasma and dialysate solute concentrations occur. This process may remove or add solute to the plasma water space depending upon the relative concentrations in dialysate and plasma. Water will also move along a gradient, in this case the osmolar or osmotic gradient, in effect “following” the solute. Diffusive clearance is more effective at removal of small solute, such as serum ions and urea, than for larger solute.

Convective clearance (hemofiltration or ultrafiltration) utilizes a pressure gradient rather than concentration gradient and has its main effect on water movement with solute movement in conjunction with water. The transmembrane pressure difference is increased as needed to “push” water through the membrane down a pressure gradient. This bulk flow of plasma water “drags” solute with it (convective mass transfer) in the formation of ultrafiltrate. Small solute removal is nearly the same as with diffusion, but fluid removal is far superior with convective clearance. Additionally, clearance of small solute is equivalent to diffusion, but convection demonstrates increased middle molecule (500-5,000 Dalton) clearance and is limited by membrane characteristics. Thus, ultrafiltration includes the generation of a pressure gradient across the permeable membrane of the dialyzer to impart fluid flow from the blood to the dialysate.

During hemodialysis, water and sodium are not ordinarily removed by diffusion but rather through the process of ultrafiltration. Ultrafiltration is commonly accomplished by lowering the hydrostatic pressure of the dialysate compartment of the dialyzer, thus allowing water containing electrolytes and other permeable substances to move from the plasma to the dialysate. For purposes of the description, the term ultrafiltration is taken to encompass the withdrawal of fluid in the dialyzer including both ultrafiltration and hemofiltration.

The present disclosure provides for improving the accuracy in measurement of the withdrawal of fluid in the dialyzer, through the measurement of blood flow into the dialyzer and blood flow out of the dialyzer. Specifically, the present system provides for calibrating, such as but not limited to matching and equalizing, blood flow measurements by a first flow sensor measuring blood flow into the dialyzer and a second flow sensor measuring blood flow out of the dialyzer so as to provide a blood side measurement system for identifying and quantifying a rate of liquid transfer into or out of the blood side of the renal replacement therapy device.

For purposes of description, it is understood the term “blood” includes treated or untreated blood, including artificial or natural blood, as well as plasma. As used herein, it is understood the term “identify” means to establish or indicate what something is, and encompasses the term “quantify,” wherein the term “quantify” means to express or measure the quantity. The ultrafiltration, which is the removal of fluid from the blood, can be measured, wherein the ultrafiltration is set forth as a rate (volume per unit time) and thus volume can be calculated by multiplying the time at the measured ultrafiltration rate.

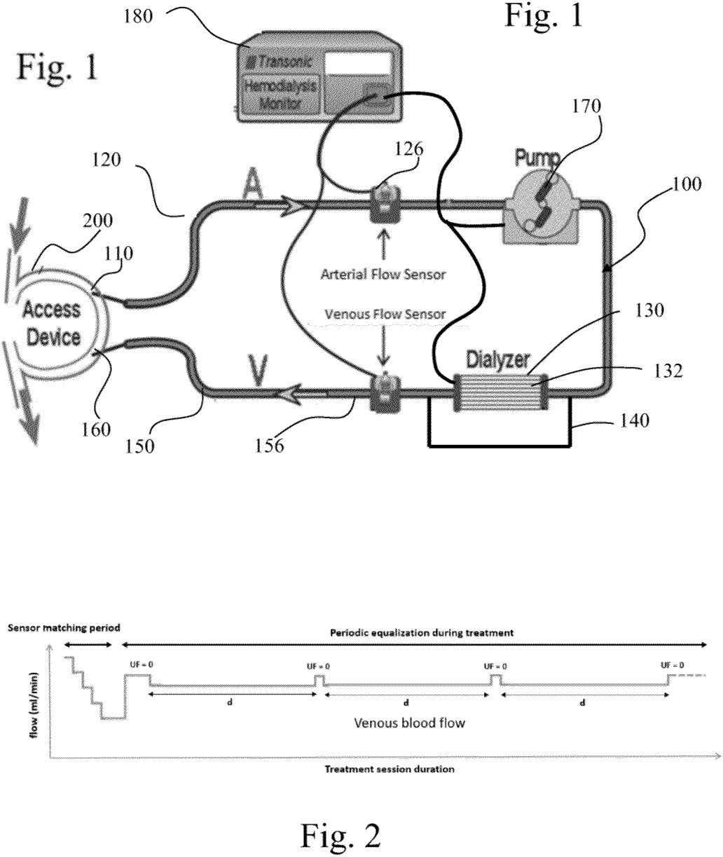

Referring to FIG. 1, an extracorporeal circuit 100 is shown connected through an access device 200 to a circulatory system of a patient. In one configuration, the extracorporeal circuit 100 provides for renal replacement therapy, wherein the extracorporeal circuit includes the renal replacement therapy device 130 having a permeable membrane 132. The renal replacement therapy includes, but is not limited to hemodialysis, hemofiltration, and hemodiafiltration.

The access device 200 fluidly connects to a circulatory system such as a human (or animal) circulatory system which includes blood, a vascular system having a cardiopulmonary system and a systemic system connecting the cardiopulmonary system to the tissues of the body, and a heart. Specifically, the systemic system passes the blood though the vascular system (arteries, veins, and capillaries) throughout a patient body. Thus, the access device 200 fluidly connects to the circulatory system and provides access to the extracorporeal circuit 100. The term “access device” encompasses any access to the circulatory system of the patient and includes but not limited to catheters, needles, shunts, AV native fistulae, AV-artificial graft; as well as a venous catheter, or other vascular implantations. The connection of the extracorporeal circuit 100 to the patient, via the access device 200, usually includes catheters or cannulas or needles, e.g. dialysis cannulas, where the access device 200, for example, is punctured and fluid communication is established. Thus, the access device 200 can include a patient blood withdrawal site 110 and a patient blood delivery site 160. As set forth herein, the access device 200 encompasses the patient blood withdrawal site 110 as well as the patient blood delivery site 160. Thus, the access device 200 includes separate arterial access and venous access as well as arterial access and venous access that are proximal or adjacent, or within a common shunt line, or graft.

The extracorporeal circuit 100 extends from the patient blood withdrawal site 110 through the renal replacement therapy device 130 and back to the patient blood delivery site 160, and includes a pump 170 configured to pump blood through the extracorporeal circuit 100 from the blood withdrawal site, through the renal replacement therapy device and to the patient blood delivery site.

The renal replacement therapy device 130 includes a blood input line 120 delivering blood from the withdrawal site 110 to the renal replacement therapy device, a permeable membrane and a blood delivery line 150 delivering blood from the renal replacement therapy device to the patient blood delivery site 160. A blood delivery line 150 connects the flow of the extracorporeal circuit 100 to the circulatory system, such as through the access device 200. The blood delivery line 150 typically includes a return cannula providing the fluid connection to the access device 200. Although the pump 170 is shown located in the extracorporeal circuit 100, it is understood the pump can be incorporated into the renal replacement therapy device 130. Similarly, the blood input line 120 and the blood delivery line 150 can be part of the extracorporeal circuit 100 or the renal replacement therapy device 130 without deviating from the scope of the present disclosure. As well known in the art, the renal replacement therapy device 130 is configured to provide a known ultrafiltration rate, such as a zero ultrafiltration rate and at least one, and in certain configurations, a plurality of target ultrafiltration rates. The zero ultrafiltration rate means that no liquid passing through the membrane 132 from the blood side to the dialysate side. Similarly, the pump 170 is configured to provide a plurality of flow rates in the extracorporeal circuit 100 and hence through the renal replacement therapy device 130.

The pump 170 can be any of a variety of pumps types, including but not limited to a peristaltic, a roller, an impeller, or a centrifugal pump. The pump 170 induces a blood flow rate through the extracorporeal circuit 100. Depending on the specific configuration, the pump 170 can be directly controlled at the pump or can be controlled through a controller 180 to establish a given blood flow rate in the extracorporeal circuit 100. The pump 170 can be at any of a variety of locations in the extracorporeal circuit 100, and is not limited to the position shown in FIG. 1. In one configuration, the pump 170 is a commercially available pump and can be set or adjusted to provide any of a variety of flow rates, wherein the pump flow rate can be read by a user and/or transmitted to and read by the controller 180. In one configuration, the pump 170 can provide a plurality of flow rates within a given range.

Depending upon the configuration of the extracorporeal circuit 100 and the mechanisms for measuring the blood parameters, the blood withdrawal line 120 can also include or provide an introduction port as a site for introducing a material into the extracorporeal circuit 100. Although not shown, it is contemplated the extracorporeal circuit 100 and specifically the blood delivery line 150 can include an air trap and air detector between the renal replacement therapy device 130 and the access device 200.

As the extracorporeal circuit 100 is configured to provide dialysis, the blood withdrawal line 120 may sometimes be referred to as an arterial line and the blood delivery line 150 may sometimes be referred to as a venous line. The “arterial line” or side is that part of the extracorporeal circuit 100 which blood passes from the patient blood withdrawal site 110, such as the access device 200 to flow to the renal replacement therapy device 130. The “venous line” or side is that part of the extracorporeal circuit 100 which blood passes from the renal replacement therapy device 130 to the patient blood delivery site 160, such as the access device 200. For purposes of description in terms of dialysis nomenclature, the blood travels from the patient blood withdrawal site 110 (in the access device 200) to the arterial line 120 (the blood withdrawal line) and returns to the patient blood delivery site 160 (in the access device) through the venous line 150 (the blood delivery line).

In the present disclosure, the term “upstream” of a given position refers to a direction against the flow of blood, and the term “downstream” of a given position is the direction of blood flow away from the given position.

A first flow sensor 126 is configured to measure flow rate in the blood withdrawal line 120 by obtaining blood flow rate data from the blood withdrawal line and a second flow sensor 156 is configured to measure flow rate in the blood delivery 150 by obtain blood flow rate data from the blood delivery line. In the dialysis nomenclature, the first flow sensor 126 obtaining flow rate data in the blood withdrawal (arterial) line 120 may sometimes be referred to as the arterial flow sensor and the second flow sensor 156 obtaining flow rate data in the blood delivery (venous) line 150 is referred to as the venous flow sensor.

The term “flow sensor” encompasses any sensing device that provides a signal representing the flow rate data or data from which the flow rate, any pulsation, variation, frequency change, or oscillation in the flow rate, or surrogate of the flow rate, pulsation, variation, frequency change, or oscillation in the flow rate can be determined, or sensed.

The normal or forward blood flow through the extracorporeal circuit 100 includes withdrawing blood through the arterial line 120 from the access device 200, passing the withdrawn blood through the extracorporeal circuit (to treat the blood in the dialyzer 130), and introducing the withdrawn (or treated) blood through the venous line 150 into the access device. The pump 170 can induce a blood flow through the extracorporeal circuit 100 from the access device 200 and back to the access device.

The first flow sensor 126 and the second flow sensor 156 are operatively coupled to the respective line and are configured to obtain flow rate data, where the term “flow rate data” is any data from which a flow rate can be derived, assessed, or calculated, as well as any surrogate data for deriving, assessing, or calculating the flow rate. It is further contemplated that the flow rate can be the actual blood flow rate, the calculated blood flow rate, or a predicted flow rate, as well as any surrogate of the actual blood flow rate, such as but not limited to a flow velocity, or a value proportional or related to the blood flow or the velocity. The flow rate data encompasses any signals or data related to the blood flow, and particularly related to any pulsatile, varying, frequency dependent, or oscillatory component or characteristic or variation of the flow, such as indicated by any signals, such as but not limited to optical signals, acoustic signals, electromagnetic signals, temperature signals and other signal that can be source of frequency analysis. Thus, the flow rate data includes any signals or data representing the flow rate or signals or data from which the flow rate, or any pulsation, variation, frequency variation, or oscillation of the flow rate, or pulsation, variation, frequency variation, or oscillation in the flow rate can be determined, or sensed, or any corresponding surrogates. For example, markers in the blood, including native or introduced particles could be used as the surrogate. Thus, the term flow rate is intended to encompass any value or measurement that corresponds to, is a surrogate of, or can represent the blood flow and especially to any pulsation, variation, frequency variation, oscillation, or a characteristic or property of the blood flow. The term “flow rate” (or “blood flow rate”) thus encompasses the volumetric flow rate as a measure of a volume of liquid passing a cross-sectional area of a conduit per unit time, and may be expressed in units of volume per unit time, typically milliliters per min (ml/min) or liters per minute (l/min), and any of its surrogates. It is understood the blood flow rate can be measured as well as calculated by any of a variety of known systems and methods. For purposes of description, measuring the flow rate encompasses obtaining or measuring the flow rate data.

Thus, the first flow sensor 126 and the second flow sensor 156 can include a flow rate sensor, an ultrasound sensor or even a dilution sensor for sensing passage of the indicator through the extracorporeal circuit 100. The first flow sensor 126 and the second flow sensor 156 can be any of a variety of sensors which obtain flow rate data. In select configurations, the first flow sensor 126 and the second flow sensor 156 can measure different blood properties: such as but not limited to temperature, Doppler frequency, electrical impedance, optical properties, density, ultrasound velocity, concentration of glucose, oxygen saturation and other blood substances (any physical, electrical or chemical blood properties). In one configuration, the first flow sensor 126 and the second flow sensor 156 are clamp on sensors that are external to the respective blood withdrawal line 120 and blood delivery line 150.

Thus, the first flow sensor 126 and the second flow sensor 156 can measure a flow characteristic or parameter to generate flow rate data, from which the flow rate, or in certain configurations flow pulsation, variation, frequency change, oscillation component, or flow frequency components can be determined. Alternatively, there can be an additional sensor (not shown) in addition to the first flow sensor 126 and the second flow sensor 156 to measure select blood characteristics or properties.

It is also understood the flow sensors 126, 156 can be located outside of the extracorporeal circuit 100. That is, the flow sensors 126, 156 can be remotely located and measure in the extracorporeal circuit 100, the changes produced in the blood from the indicator introduction or values related to the indicator introduction which can be transmitted or transferred by means of diffusion, electro-magnetic or thermal fields or by other means to the respective sensor.

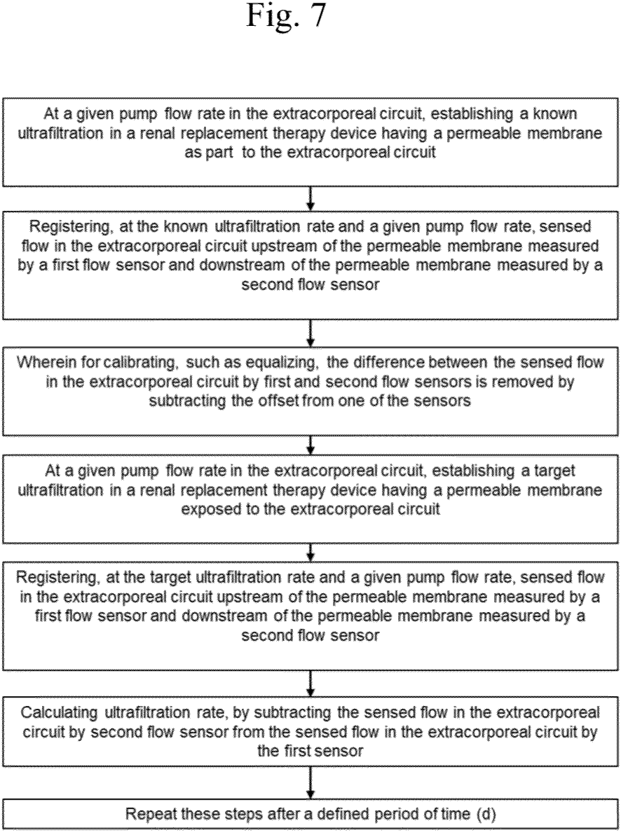

The controller 180 is connected to the flow sensors 126, 156, and can be connected to the pump 170 as well as the renal replacement therapy device 130. The term “controller” includes signal processors and computers, including programmed desk or laptop computers, or dedicated computers for processors. Such controllers 180 can be readily programmed to perform the recited calculations, or derivations thereof, to provide determinations of the flow rate and transforms of the flow rate data as set forth herein, and seen for example in FIG. 6. The controller 180 can also perform preliminary signal conditioning such as summing one signal with another signal or portion of another signal. The controller 180 can be a stand-alone device such as a personal computer, a dedicated device or embedded in one of the components, such as the pump 170 or the renal replacement therapy device 130. The controller 180 can include or be operably connected to a memory, as well as an input/output device such as a touch screen or keypad or keyboard as known in the industry. Although the controller 180 is shown as connected to the first and second flow sensors 126, 156, the pump 170, and the renal replacement therapy device 130, it is understood the controller can be connected to the flow sensors, or the flow sensors and the pump, or any combination of the flow sensors, the pump, and the renal replacement therapy device.

The present method and apparatus provide for measuring blood flow upstream (Q_{H}) of the renal replacement therapy device 130 (such as a dialyzer) and blood flow downstream (Q_{V}) of the renal replacement therapy device and based on the difference between blood flow into and out of the renal replacement therapy device, assessing fluid removal (ultrafiltration) during the blood treatment session (HD) session, wherein the ultrafiltration can be measured substantially continuously during the session. Specifically, the present disclosure is directed to an issue in current approaches of addressing errors in blood flow measurement systems with clamp-on sensors, wherein the errors are much greater than the necessary accuracy of measurements for determining fluid removal by the renal replacement therapy device 130.

Sources of errors by clamp-on flow sensors include, but are not limited to, factory calibration, inconsistency of tubing that the flow sensors were calibrated on the factory versus in the field tubing, fluctuations of temperature between measurements, different characteristics of the arterial and the venous flow sensor across the flow range of the renal replacement therapy device 130 (and/or the pump 170), in that the differences may be larger at higher flows and smaller at lower flows, or vice versa.

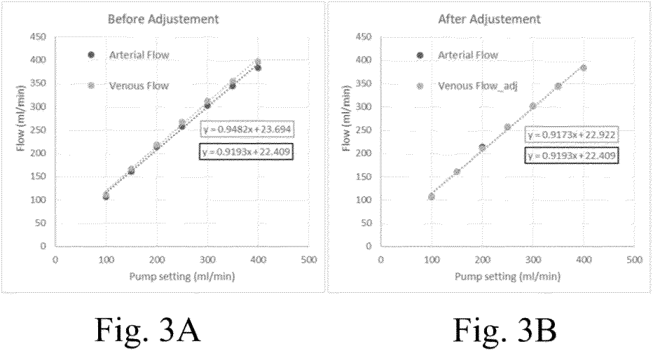

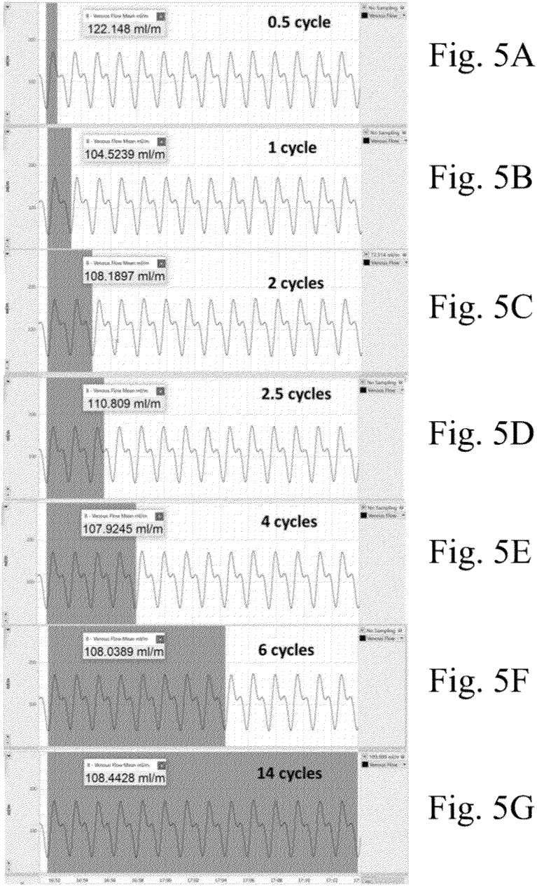

The present system is directed to reducing the error in the measurements of the flow sensors on the arterial and venous blood lines 120, 150 by calibrating, such as matching, the arterial and venous flow sensors 126, 156 from a plurality of flow comparisons in the extracorporeal circuit 100 which can accommodate current tubing and current patient conditions in real time, as well as providing for the periodic calibration, such as equalization, throughout the treatment session. The calibration of the flow sensors 126, 156 encompasses matching the first flow sensor 126 and the second flow sensor 156 either one to the other or to a common point. It is further contemplated the calibration can encompass an equalization of the signals from the first flow sensor 126 and the second flow sensor 156 for a common flow at the known ultrafiltration, wherein the signals from at least one of the flow sensors are adjusted, such as to be equal. As seen in FIG. 2, the calibration can encompass matching the first and the second flow sensors 126, 156 as well as equalizing the first and the second flow sensors. As seen in FIG. 4, an offset between the first and the second flow sensors is eliminated by the equalization.

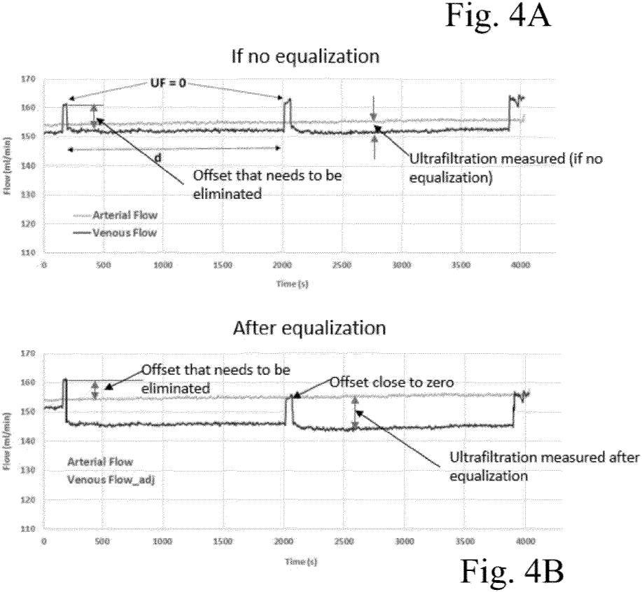

In a treatment session, a patient is fluidly connected to the extracorporeal circuit 100. Prior to treatment of the blood, such as ultrafiltration, by the renal replacement therapy device 130, a blood flow is established through the extracorporeal circuit 100. Thus, the treatment session includes the time the patient is operably connected to the extracorporeal circuit 100, and the blood is merely flowing through the extracorporeal circuit untreated, as well as the time the blood is being treated by the renal replacement therapy device 130, such as ultrafiltration. The treatment session may be 2 hours, 3 hours, four hours, or longer, and the session is typically dominated by blood treatment time. In one configuration, at step 1, typically during the treatment session but before starting the blood treatment process by the renal replacement therapy device 130 (such as the ultrafiltration process) when blood is passing through the extracorporeal circuit 100 (including passing through the blood input line 120 and the blood delivery line 150) and the ultrafiltration rate is known, such as zero, the blood flow in the renal replacement therapy device (and hence extracorporeal circuit 100) is changed, such as by the controller 180. In one configuration, the change or range of blood flows through the extracorporeal circuit 100 (including the renal replacement therapy device 130) is within an expected range of blood flow during the treatment (or within a given extended percentage, such as +/- 5%, or +/- 10% or +/- 20% of the expected range). The measured flow rates (the corresponding generated signals) by each of the first flow sensor 126 and the second flow sensor 156 during the known (or zero) ultrafiltration rate are registered, such as received, by the controller 180. The registered signals (measured flow rates) from the respective flow sensors can be stored or recorded by the controller 180 and in one configuration plotted against the pump flow to provide a corresponding curve having a slope. The slopes of the arterial and venous flow sensors 126, 156 are then calibrated by matching as shown in FIG. 3. Alternatively, to obtain the effective known (or zero ultrafiltration rate), the extracorporeal circuit 100 and/or the renal replacement device 130 can include a bypass line 140 for the blood flow to bypass exposure to the semi-permeable membrane. In either configuration, (the known ultrafiltration rate, such as the zero ultrafiltration rate or the bypass, here collectively taken as known ultrafiltration rate), the actual flow through the first flow sensor in the withdrawal line 120 must be the same as the flow through the delivery line 150, By calibrating, such as matching, adjusting (using a correction factor), or through a lookup table, the first flow sensor 126 and the second flow sensor 156 at common flow rates, deviations between the flow sensors can be accounted for, thereby improving the accuracy of the respective flow measurements through the blood withdrawal (arterial) line 120 and the blood delivery (venous) 130 during a treatment session having a given ultrafiltration rate. That is, at a given common flow typically within the expected operating range and at the known ultrafiltration rate, the first flow sensor 126 and the second flow sensor 156 will provide an equal measurement of the flow by virtue of their calibration. As set forth herein, the calibration can include the controller 180 processing of the signal received from the flow sensors 126, 156.

By obtaining measured flow rates at a minimum of two different flow rates in the calibration process, the first flow sensor 126 and the second flow sensor 156 can be calibrated, such as matched. It is understood that a plurality of different flow rates can be imparted through the extracorporeal circuit 100 and the corresponding flow measurements of the first flow sensor 120 and the second flow sensor 156 used to calibrate the sensors to each other. Thus, additional flow measurements at the known (such as zero) ultrafiltration rate can be used in the calibration of the sensors such as by providing additional data points in the corresponding curves or data points in the lookup tables. As more data is obtained, the controller 180 can employ a curve fitting algorithm, such as an adjusting factor known in the art, to accommodate the additional data.

Calibrating the first flow sensor 126 and the second flow sensor 156 can include matching such as graphing the actual flow rate against the measured flow rate for each of the sensors at a first and a second flow rate in the extracorporeal circuit 100, at the known (such as zero) ultrafiltration rate. Each of the first flow sensor 126 and the second flow sensor 156 then has an associated curve (relating the respective measured flow rate to the actual or pump flow rate), and the curves can be matched for measuring flow rate during ultrafiltration, such as for example, interpolating from the respective curve or adjusting or changing the curve of one of the flow sensors to match the curve of the remaining flow sensor.

Alternatively, the calibrating can include adjusting the flow rate data from one of the first flow sensor 126 and the second flow sensor 156 at a given flow rate to correspond to, or match, or be equal to, the flow rate data of the remaining one of the first flow sensor and the second flow sensor.

Further, the calibrating can include mechanically adjusting the respective flow sensor or the associated signal at the given flow rate, so that the resulting measured flow rate of the first flow 126 sensor and second flow sensor 156 are equal. That is, if the respective flow sensor has a mechanical adjustment, calibration or tuning, the flow sensor can be matched to the remaining flow sensor, or the actual flow at the known (such as zero) ultrafiltration.

It is further understood the calibrating can be accomplished by applying a lookup table for the flow rate data obtained by at least one of the first flow sensor 126 and the second flow sensor 156 at the respective flow rates during the known ultrafiltration. The lookup table can be any array that provides an indexing operation, such as index mapping. The lookup table includes an array or matrix of data that contains items that are searched. The lookup table can be arranged as key-value pairs, where the keys are the data items being searched (looked up) and the values are either the actual data or pointers to where the data are located.

In one configuration, the calibration to provide matching of the first flow sensor 126 and the second flow sensor 156 is done during the treatment session, prior to any ultrafiltration. However, it is understood the matching can be performed after, or even during the treatment session, where the previously obtained flow rate data from the first flow sensor 126 and the second flow sensor 156 is then adjusted corresponding to the matching of the flow sensors. However, it is anticipated that matching the first flow sensor 126 and the second flow sensor 156 during the treatment session, but prior to the blood treatment, provides advantages of reducing data processing as well as providing real time values for guiding the treatment.

In addition to calibrating, such as matching the first flow sensor 126 and the second flow sensor 156 before starting the treatment session, a periodic calibration of the flow sensors, such as equalization, can be performed to accommodate potential changes over time during a treatment session. The ultrafiltration rate of the renal replacement therapy device 130 can be periodically set to a known ultrafiltration rate, such as zero, and for at least one blood flow rate through the extracorporeal circuit 100, wherein the measurements of the first flow sensor 126 and the second flow sensor 156 are measured and then calibrated by being equalized. The measurement of the first flow sensor 126 can be adjusted to the measurement of the second flow sensor 156, or the measurement of the second flow sensor can be adjusted to the measurement of the first flow sensor, or the measurement of each of the first flow sensor and the second flow sensor can be set to a different equal number.

It is contemplated that at the periodic times during the treatment session, the ultrafiltration rate is set to a known rate, such as zero, and at least one common flow rate is exposed to the first flow sensor 126 and the second flow sensor 156, wherein the first and the second flow sensors are calibrating by equalizing. It is understood that a plurality of different common flow rates can be exposed to the first flow sensor 126 and the second flow sensor 156 during the time of the known ultrafiltration rate. From this plurality of measurements, the first flow sensor 126 and the second flow sensor 156 are calibrated, such as matched or equalized. This periodic interval of known (or zero) ultrafiltration rate can be applied by the controller 180 at predetermined times, or at predetermined thresholds or triggers, or manually initiated. Thus, at predetermined periods or intervals within a treatment session, the ultrafiltration rate can be set to a known rate, such as zero, and the resulting flow rate data from the first flow sensor 126 and the second flow sensor 156 is used to identify any offset between the first flow sensor and the second flow sensor, as shown in FIG. 3, so that the flow sensors are calibrated by being equalized by the removal of the offset.

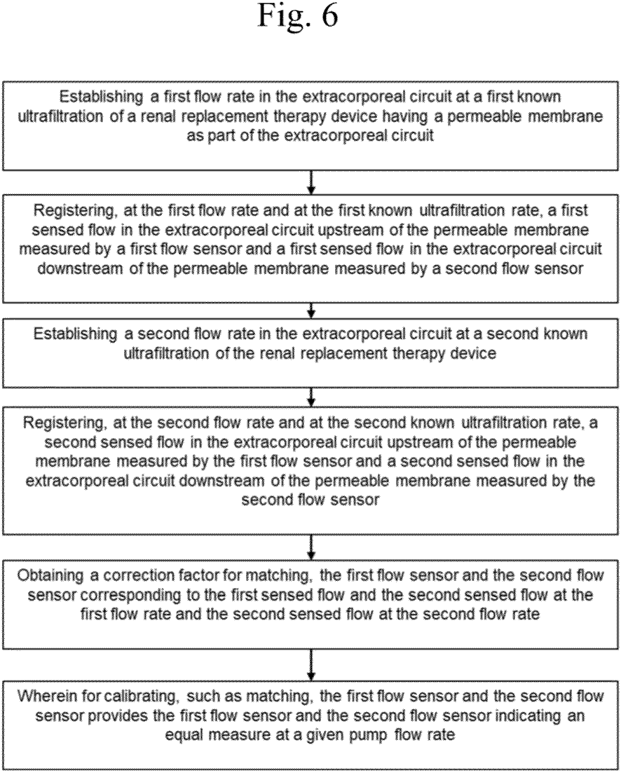

A further aspect of the present disclosure relates to the collection of the flow rate data. Specifically, with respect to obtaining the flow rate data, the flow rate in the blood withdrawal line 120 and the blood delivery line 150 is generally pulsatile. Therefore, an instantaneous reading or data representing less than a full pulse cycle (or a few cycles) while accurate of that moment does not provide the necessary average flow rate. To address the pulsatile component of the flow rate in the blood withdrawal line 120 and the blood delivery line 150, the collection of the flow rate data is taken over a sufficient time to mitigate errors introduced into the measured flows from the pulsatile component. In one configuration, a collection of flow rate data over a period of at least 2 cycles has been found satisfactory. Alternatively, the pulsatile flow can be accommodated by choosing the same flow point in a respective cycle as the start and the finish period of averaging, as shown in FIG. 5.

By increasing the accuracy of the blood flow measurement (of the flow in the input line 120 and the delivery line 150), the contribution from the renal replacement therapy device 130 can be determined with corresponding accuracy. Specifically, Qₐ= Qᵥ + Q_{UF}

Where Qᵥ is the flow rate in the blood delivery line 150,

Qₐ is the flow rate in the blood withdraw line 120, and

Q_{UF} is the flow rate from ultrafiltration of the renal replacement therapy device 130, that is Q_{UF} is the ultrafiltration rate.

Therefore, Q_{UF} = Qa - Qv. By providing the measurements of Qᵥ and Qₐ within known or acceptable accuracy parameters, the accuracy of the ultrafiltration rate Q_{UF} can be thus known and relied upon.

By knowing the ultrafiltration (rate), the volume, or amount, of fluid removal can be obtained: UF volume (ml) = Q_{UF} * T, where Q_{UF} is the flow rate from ultrafiltration of the renal replacement therapy device 130, that is Q_{UF} is the ultrafiltration rate in ml/min unit, and T is the duration which ultrafiltration was turned on (the time period that UF ≠ 0).

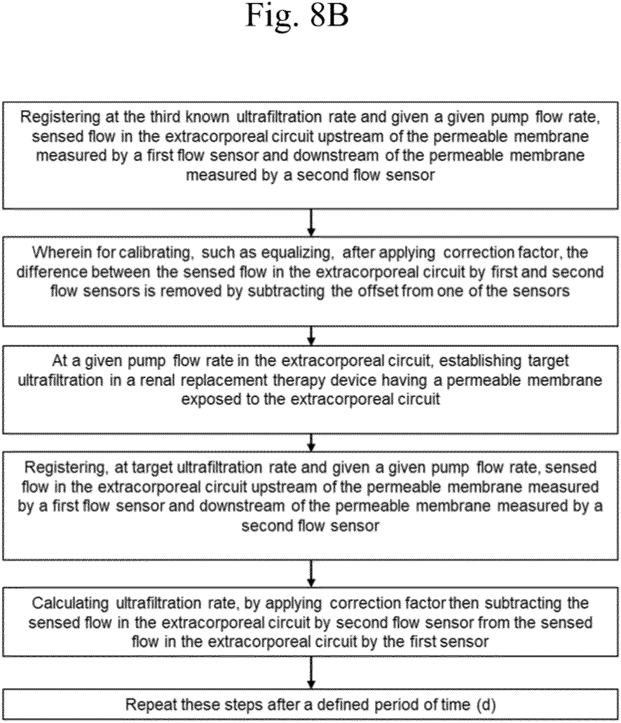

The controller 180 is programmed to calibrate, such as match, the first flow sensor 126 and the second flow sensor 156 by any of the set forth mechanisms, as well as any equivalent. Further, the controller 180 can calculate the ultrafiltration rate Q_{UF} from the measured flow rates in the blood withdraw line 120 and the blood delivery line 150 from the respective first flow sensor 126 and the second flow sensor 156. From the calculated ultrafiltration rate, the volume of fluid removal as set forth above. Referring to FIGS. 8A and 8B, the controller can be configured to calibrate the first flow sensor 126 and the second flow sensor 156 by matching and equalizing during a given treatment session.

The controller 180 can be configured to maintain a blood flow through the extracorporeal circuit 100 while temporarily stopping ultrafiltration during the treatment session to provide a known ultrafiltration rate, register a flow rate from the first flow sensor 126 and the second flow sensor 156 during the time of the known ultrafiltration rate, re-calibrate the first flow sensor and the second flow sensor corresponding to the registered flow rate from the first flow sensor and the second flow sensor, initiate ultrafiltration and register a flow rate from the re-calibrated first flow sensor and the second flow sensor, and determine the ultrafiltration rate corresponding to the flow rate from the re-calibrated first flow sensor and the second flow sensor.

Thus, the present disclosure provides a method of measuring ultrafiltration rate and cumulative ultrafiltration, by the renal replacement therapy device 130 in the extracorporeal circuit 100 having the pump 170, the renal replacement therapy device configured to provide a first and a second known (such as zero) ultrafiltration rate and a target ultrafiltration rate, and having the blood input line 120 delivering blood to the renal replacement therapy device, the permeable membrane 132, and the blood output line 150 passing blood from the renal replacement therapy device, the method including the steps of (a) registering, during a first pump flow rate and the first known (such as zero) ultrafiltration rate, a first difference between a first flow rate in the blood input line measured by the first flow sensor 126 and a first flow rate in the blood output line measured by the second flow sensor 156; (b) registering, during a second pump flow rate and the second known (such as zero) ultrafiltration rate, a second difference between a second flow rate in the blood input line measured by the first flow sensor and a second flow rate in the blood output line measured by the second flow sensor; (c) calibrating, such as matching, the first flow sensor and the second flow sensor corresponding to the first difference and the second difference; and (d) measuring or deriving the target ultrafiltration rate at a target pump flow rate from a third flow measurement from the calibrated first flow sensor and second flow sensor. As set forth above, it is contemplated the calibrating can include matching the slopes from curves associated with the first and second measured flow rates of each of the first and second flow sensors. In addition, the first known ultrafiltration rate and the second known ultrafiltration rate can be equal, and can as well be a zero ultrafiltration rate.

In a specific application, the present disclosure provides a method of measuring ultrafiltration by the renal replacement therapy device 130, the renal replacement therapy device configured to provide a known, such as a zero ultrafiltration rate and a target ultrafiltration rate, in an extracorporeal circuit 100 having the blood input line 120 delivering blood to the renal replacement therapy device, the pump 170, the permeable membrane 132, and the blood output line 150 passing blood from the renal replacement therapy device, the method including the steps of (i) calibrating, such as matching, prior to ultrafiltration in a treatment session, the first flow sensor 126 measuring blood flow in the blood input line and the second flow sensor 156 measuring blood flow in the blood delivery line; (ii) recalibrating, such as equalizing, at a known or zero ultrafiltration rate during the treatment session, the first flow sensor and second flow sensor; and (iii) assessing a target ultrafiltration rate based on flow measurements by the recalibrated first flow sensor and the second flow sensor. The disclosure further provides for estimating an amount of fluid removal by ultrafiltration during the treatment session corresponding to the assessed target ultrafiltration rate. Additional steps can include averaging the flow measurement of the first flow sensor 126 and the second flow sensor 156 over a plurality of pulses within the measured flow or taking the flow measurements across a common point within a flow pulse, to eliminate flow measurement error causing by fluctuation in pulsations in the measured flow. It is further contemplated the calibrating, such as equalizing steps can be sufficient to accommodate drift/variance between the flow sensors 126, 156 occurring during the treatment session. It is understood the calibrating, such as matching or equalizing, can be done periodically throughout the treatment session, wherein the assessing is based on a difference between calibrated flow measurements from the first flow sensor 126 and the second flow sensor 156.

The present disclosure provides a further method of measuring a target ultrafiltration rate by the renal replacement therapy device 130, the renal replacement therapy device configured to provide a known, such as zero ultrafiltration rate and the target ultrafiltration rate, and operably connected to the blood input line 120 delivering blood to the renal replacement therapy device, the pump 170, the permeable membrane 132, and the blood output line 150 passing blood from the renal replacement therapy device, the method including the steps of (a) calibrating the first flow sensor 126 configured to sense a flow in the blood input line and the second flow sensor 156 configured to sense flow in the blood output line, and (b) deriving/calculating the target ultrafiltration rate based on measured flow rates from the calibrated first flow sensor and the second flow sensor, wherein the calibrating includes at least one of:
(i) a first flow rate measured by each of the first flow sensor and the second flow sensor at a first pump flow rate and a first known ultrafiltration rate, and (ii) a second flow rate measured by each of the first flow sensor and the second flow sensor at a second pump flow rate and a second known ultrafiltration rate; and (b) identifying slopes of curves of the measured first flow rate and second flow rate against flow standards (or the pump flow) for each of the first flow sensor and the second flow sensor and matching the slopes;
(ii) periodically during the treatment session equalizing, the first flow sensor and the second flow sensor at a known ultrafiltration rate;
(iii) periodically during the treatment session equalizing the first flow sensor and the second flow sensor, wherein the equalizing is derived from an offset identified between the first flow sensor and the second flow sensor at a given pump flow rate and a known ultrafiltration rate, and adding or subtracting the offset from a subsequent flow measurement of one of the first flow sensor and the second flow sensors; and
(iv) averaging flow measurement of the first flow sensor and second flow sensor over a sufficient period to encompass at least one pulsatile cycle within the flow or measuring common start and finish point of a pulsatile cycle within the flow.

An additional disclosed method includes measuring an ultrafiltration rate by a renal replacement therapy device 130 in a system having a pump, the renal replacement therapy device configured to provide a known, such as zero, ultrafiltration rate and a target ultrafiltration rate and having a blood input line delivering blood to the renal replacement therapy device, a permeable membrane, and a blood output line passing blood from the renal replacement therapy device, the method including the steps of (a) obtaining, during a first pump flow rate and the known ultrafiltration rate, (i) a measured first flow rate in the blood input line and (ii) a measured first flow rate in the blood output line; (b) obtaining, during a second pump flow rate and the target ultrafiltration rate, (i) a measured second flow rate in the blood input line and (ii) a measured second flow rate in the blood output line; and (c) calculating an ultrafiltration rate corresponding to at least the measured first flow rate in the blood input line, the measured first flow rate in the blood output line, the measured second flow rate in the blood input line, and the measured second flow rate in the blood output line. The method can include the additional step of initially calibrating, such as matching, at the known ultrafiltration rate (i) the measured first flow rate in the blood input line and the measured first flow rate in the blood output line, and (ii) the measured second flow rate in the blood input line and the measured second flow rate in the blood output line.

Also provided is an alternative method of measuring an ultrafiltration rate by the renal replacement therapy device 130, the renal replacement therapy device connected to the pump 170 and configured to provide a known (such as zero) ultrafiltration rate and a target ultrafiltration rate and having the blood input line 120 delivering blood to the renal replacement therapy device, the permeable membrane 132, and the blood output line 150 passing blood from the renal replacement therapy device, wherein the method includes the steps of (a) equalizing, the first flow sensor 126 configured to measure a flow in the blood input line and the second flow sensor 156 configured to measure a flow in the blood output line; (b) operating the replacement therapy device 130 at the target ultrafiltration rate and obtaining a first treatment flow measurement from the first flow sensor and a second treatment flow measurement from the second flow sensor; and (c) deriving a value of the target ultrafiltration rate based on at least one of the equalized first flow sensor and second flow sensor, the first treatment flow measurement and the second treatment flow measurement.

Depending upon the calibration used, the present disclosure provides an apparatus for measuring an ultrafiltration rate of the renal replacement therapy device 130 in an extracorporeal circuit 100 having the pump 170, the renal replacement therapy device configured to establish a first and a second known ultrafiltration rate and a target ultrafiltration rate, the renal replacement therapy device having the blood input line 120 delivering blood to the renal replacement therapy device, the permeable membrane 132, the blood output line 150 passing blood from the renal replacement therapy device, wherein the apparatus includes (a) the first flow sensor 126 configured to obtain a blood input line flow rate data in the blood input line; (b) the second flow sensor 156 configured to measure a blood output line flow rate data in the blood output line; and (c) the controller 180 connected to the first flow sensor and the second flow sensor, the controller configured to (i) register, at a first pump flow rate and the first known ultrafiltration rate, a first difference between a first flow rate measured by the first flow sensor and a first flow rate measured by the second flow sensor and (ii) register, at a second pump rate and the second known ultrafiltration rate, a second difference between a second flow rate measured by the first flow sensor and a second flow rate measured by the second flow sensor, and (iii) identify the target ultrafiltration rate corresponding to the first difference and the second difference. It is contemplated the second flow rate and first flow rate may be the same. Similarly, it is contemplated the first known ultrafiltration rate and the second known ultrafiltration rate can be equal, including a value of zero.

Thus, the present disclosure provides apparatus for measuring an ultrafiltration rate of the renal replacement therapy device 130 in the extracorporeal circuit 100 having the pump 170, the renal replacement therapy device configured to establish a first known ultrafiltration rate, a second known ultrafiltration rate, and a target ultrafiltration rate, the renal replacement therapy device having the blood input line 120 delivering blood to the renal replacement therapy device, the permeable membrane 132, the blood output, or delivery, line 150 passing blood from the renal replacement therapy device, the apparatus including the first flow sensor 126 configured to measure a flow rate in the blood input line, the second flow sensor 156 configured to measure a flow rate in the blood output line; and the controller 180 connected to the first flow sensor and the second flow sensor, the controller configured to (i) register a first flow rate measured by the first flow sensor and a first flow rate measured by the second flow sensor each at a first pump rate and the first known ultrafiltration rate, (ii) register a second flow rate measured by the first flow sensor and a second flow rate measured by the second flow sensor each at a second pump rate and the second known ultrafiltration rate, (iii) calibrate at least one of the first flow sensor and the second flow sensor corresponding to the first flow rate measured by the first flow sensor, the first flow rate measured by the second flow sensor, the second flow rate measured by the first flow sensor, and the second flow rate measured by the second flow sensor, and (iv) identify the target ultrafiltration rate from a third flow rate measured by the first flow sensor and a third flow rate measured by the second flow sensor during the target ultrafiltration rate.

It is contemplated the controller 180 is configured to calibrate the first flow sensor and the second flow sensor with a lookup table. Further, the controller 180 can be configured to calculate (i) a first curve corresponding to the first flow rate and the second flow rate measured by the first flow sensor and (ii) a second curve corresponding to the first flow rate and the second flow rate measured by the second flow sensor. The controller 180 can be configured to match the first curve and the second curve, wherein the matching includes adjusting one of the first curve and the second curve to a remaining one of the first curve and the second curve or fitting one of the first curve and the second curve to a remaining one of the first curve and the second curve. The controller 180 can be further configured to quantify the target ultrafiltration rate. As set forth above, the extracorporeal circuit 100 can include the bypass line 140 selectively bypassing the renal replacement therapy device 130. The controller 180 can be configured to register the first flow rate measured by the first flow sensor 126 and the first flow rate measured by the second flow sensor 156 each at the first pump rate and the first and second known ultrafiltration rate over a sufficient period of data collection for the flow measurement to include at least one full cycle of pulsatile flow signal. It is also understood the controller 180 can be configured to register the first flow rate measured by the first flow sensor 126 and the first flow rate measured by the second flow sensor 156 each at a first pump rate and the first known ultrafiltration rate at a common point within a pulsatile cycle. The controller 180 can be configured to average the first flow rate measured by the first flow sensor 126 and the first flow rate measured by the second flow sensor 156 each at a first pump rate and the respective known ultrafiltration rate over a sufficient period of registered flow to include at least one full cycle of pulsatile flow signal within the average. Also, the controller 180 can be configured to employ the first known ultrafiltration rate and/or the second known ultrafiltration rate as equal rates, including a zero ultrafiltration rate.

The present disclosure provides a method of measuring a target ultrafiltration rate by the renal replacement therapy device 130 in the extracorporeal circuit 100 having the pump 170, the renal replacement therapy device configured to provide a first and a second known ultrafiltration rate and the target ultrafiltration rate, and having the blood input line 120 delivering blood to the renal replacement therapy device, the permeable membrane 132, and the blood output line 150 passing blood from the renal replacement therapy device, the method including (a) measuring, at a first pump flow rate and the first known ultrafiltration rate, (i) a first flow rate in the blood input line by the first flow sensor 126 and (ii) a first flow rate in the blood output line by the second flow sensor 156; (b) measuring, at a second pump flow rate and the second known ultrafiltration rate, (i) a second flow rate in the blood input line by the first flow sensor and (ii) a second flow rate in the blood output line by the second flow sensor; (c) matching at least one of the first flow sensor and the second flow sensor corresponding to the first flow rate in the blood input line measured by the first flow sensor, the first flow rate in the blood output line measured by the second flow sensor, the second flow rate in the blood input line measured by the first flow: sensor, and the second flow rate in the blood output line measured by the second flow sensor; (d) measuring, during the target ultrafiltration rate and a target pump flow rate, (i) a third blood flow rate in the blood input line by the matched first flow sensor and (ii) a third blood flow rate in the blood output line flow by the matched second flow sensor; and (e) identifying the target ultrafiltration rate corresponding to the third blood flow rate in the blood input line measured by the matched first flow sensor and the third blood flow rate in the blood output line flow measured by the calibrated second flow sensor. The method can further include, after step (e), (f) establishing a known ultrafiltration rate and a given blood flow rate through the blood input line and the blood output line; (g) identifying an offset between a first measurement of the given blood flow rate in the blood input line 120 by the first flow sensor 126 and a second measurement of the given blood flow rate in the blood output line 150 by the second flow sensor 156; and (h) adjusting, by the offset, a subsequent flow measurement of at least one of the first flow sensor and the second flow sensor. That is, the method can further include equalizing, at least one of the first flow sensor and the second flow sensor corresponding to the flow measured by the first flow sensor and second flow sensor during a known ultrafiltration rate and a target pump flow rate. It is understood the target pump rate can be one of the first pump rate and the second pump rate. In addition, calibrating the first flow sensor and the second flow sensor includes adjusting at least one of the first flow sensor and the second flow sensor. In the method, matching the first flow sensor and the second flow sensor can include applying a lookup table. In the method, identifying the target ultrafiltration rate can include quantifying the target ultrafiltration rate. Further, the known ultrafiltration rate can be a zero ultrafiltration rate and can be obtained by turning off the ultrafiltration in the renal replacement therapy device 130 or bypassing the renal replacement therapy device. In the method a controller can be configured to average over a sufficient period of registered flow to include at least one full cycle of pulsatile flow signal within the averaging to reduce, or eliminate, flow measurement error from the fluctuation in pulsations.

While the description is set forth as a renal replacement device 130, the present disclosure encompasses any device for “blood treatment” such as but not limited to any blood processing including but not limited to dialysis, which in turn includes toxin clearance such as by diffusive as well as conductive therapy including but not limited to hemofiltration, hemodialysis, hemodiafiltration, or Continuous Renal Replacement Therapy (CRRT). The renal replacement therapy device includes a blood treatment device such as any device for imparting the blood treatment. Thus, in one configuration, the blood treatment device, such as the dialyzer, can be configured to provide controllable transfer of solutes and water across a semi permeable membrane separating flowing blood and dialysate streams. Such a transfer process may include diffusion (dialysis) and convection (ultra-filtration). The blood treatment device may provide any of a host of other blood treatments, such as chemical treatment, electromagnetic treatment as well as thermal treatment. Though the present disclosure is set forth in terms of dialysis in extracorporeal renal replacement therapy renal replacement therapy device, it is understood this includes hemodialysis, hemofiltration, and hemodiafiltration.

This disclosure has been described in detail with particular reference to an embodiment, but it will be understood that variations and modifications can be effected within the spirit and scope of the disclosure. The presently disclosed embodiments are therefore considered in all respects to be illustrative and not restrictive. The scope of the invention is indicated by the appended claims, and all changes that come within the meaning and range of equivalents thereof are intended to be embraced therein.

1. An apparatus for measuring an ultrafiltration rate of a renal replacement therapy device in an extracorporeal circuit having a pump, the renal replacement therapy device configured to establish a first known ultrafiltration rate, a second known ultrafiltration rate and a target ultrafiltration rate, the renal replacement therapy device having a blood input line delivering blood to the renal replacement therapy device, a permeable membrane, a blood output line passing blood from the renal replacement therapy device, the apparatus comprising:
(a) a first flow sensor configured to measure a flow rate in the blood input line;
(b) a second flow sensor configured to measure a flow rate in the blood output line; and
(c) a controller connected to the first flow sensor and the second flow sensor, the controller configured to (i) register a first flow rate measured by the first flow sensor and a first flow rate measured by the second flow sensor each at a first pump rate and the first known ultrafiltration rate, (ii) register a second flow rate measured by the first flow sensor and a second flow rate measured by the second flow sensor each at a second pump rate and the second known ultrafiltration rate, (iii) calibrate at least one of the first flow sensor and the second flow sensor corresponding to the first flow rate measured by the first flow sensor, the first flow rate measured by the second flow sensor, the second flow rate measured by the first flow sensor, and the second flow rate measured by the second flow sensor, and (iv) identify the target ultrafiltration rate from a third flow rate measured by the first flow sensor and a third flow rate measured by the second flow sensor during the target ultrafiltration rate.

2. The apparatus of claim 1, wherein the controller is further configured to periodically establish a given known ultrafiltration rate and register a fourth flow rate measured by the first flow sensor and a fourth flow rate measured by the second flow sensor, and identify a subsequent measure of the target ultrafiltration rate at least partly corresponding to the fourth flow rate measured by the first flow sensor and the fourth flow rate measured by the second flow sensor.

3. The apparatus of claim 2, wherein the controller is further configured to register the fourth flow rate measured by the first flow sensor and the fourth flow rate measured by the second flow sensor over a sufficient period of data collection for each measured flow to include at least one full cycle of a pulsatile flow signal of the measured flow.

4. The apparatus of claim 1, wherein the controller is configured to register the first flow rate measured by the first flow sensor and the first flow rate measured by the second flow sensor over a sufficient period of data collection for the flow measurement to include at least one full cycle of pulsatile flow signal.

5. The apparatus of claim 1, wherein the controller is configured to employ the respective known ultrafiltration rate as a zero ultrafiltration rate.

6. The apparatus of claim 1, wherein the controller is configured to average the first flow rate measured by the first flow sensor and the first flow rate measured by the second flow sensor each at a first pump rate and the known ultrafiltration rate over sufficient period of registered flow to include at least one full cycle of pulsatile flow signal within the average.

7. An apparatus for measuring an ultrafiltration rate of a renal replacement therapy device in an extracorporeal circuit having a pump, the renal replacement therapy device configured to establish a known ultrafiltration rate and a target ultrafiltration rate, the renal replacement therapy device having a blood input line delivering blood to the renal replacement therapy device, a permeable membrane, a blood output line passing blood from the renal replacement therapy device, the apparatus comprising:
(a) a first flow sensor configured to measure a flow rate in the blood input line;
(b) a second flow sensor configured to measure a flow rate in the blood output line; and
(c) a controller connected to the first flow sensor, the second flow sensor, and the renal replacement therapy device, the controller configured to (i) temporarily impart a known ultrafiltration rate during a treatment session, (ii) identify an offset between the first and the second flow sensor; and (iii) adjust a subsequent flow measurement of at least one of the first flow sensor and the second flow sensors by the offset.

8. The apparatus of claim 7, wherein the controller is configured to periodically provide the known ultrafiltration rate as a zero ultrafiltration rate.

9. The apparatus of claim 7, wherein temporarily imparting the known ultrafiltration rate is between 1 second and 360 seconds.

10. The apparatus of claim 7, wherein the controller is configured to (i) register a first flow rate measured by the first flow sensor and a first flow rate measured by the second flow sensor each at a first pump rate and a first known ultrafiltration rate, (ii) register a second flow rate measured by the first flow sensor and a second flow rate measured by the second flow sensor each at a second pump rate and a second known ultrafiltration rate, (iii) calibrate at least one of the first flow sensor and the second flow sensor corresponding to the first flow rate measured by the first flow sensor, the first flow rate measured by the second flow sensor, the second flow rate measured by the first flow sensor, and the second flow rate measured by the second flow sensor, and (iv) identify the target ultrafiltration rate from a third flow rate measured by the first flow sensor and a third flow rate measured by the second flow sensor during the target ultrafiltration rate.

11. The apparatus of claim 7, wherein the controller is configured to average the first flow rate measured by the first flow sensor and the first flow rate measured by the second flow sensor each at a first pump rate and the known ultrafiltration rate over sufficient period of registered flow to include at least one full cycle of pulsatile flow signal within the average.

12. A method of measuring a target ultrafiltration rate by a renal replacement therapy device in an extracorporeal circuit having a pump, the renal replacement therapy device configured to provide a first known ultrafiltration rate, a second known ultrafiltration rate, and the target ultrafiltration rate, and having a blood input line delivering blood to the renal replacement therapy device, a permeable membrane, and a blood output line passing blood from the renal replacement therapy device, the method comprising:
(a) calibrating a first flow sensor configured to sense a flow rate in the blood input line and a second flow sensor configured to sense a flow rate in the blood output line, wherein the calibrating corresponds to at least (i) a first flow rate in the extracorporeal circuit measured by each of the first flow sensor and the second flow sensor at a first pump flow rate and the first known ultrafiltration rate, and (ii) a second flow rate in the extracorporeal circuit measured by each of the first flow sensor and the second flow sensor at a second pump flow rate and the second known ultrafiltration rate; and
(b) identifying the target ultrafiltration rate corresponding to a third flow rate measured by the calibrated first flow sensor and the second flow sensor at a given pump flow rate and the target ultrafiltration rate.

13. The method of claim 12, further comprising (i) periodically establishing, during the treatment session, a second known ultrafiltration rate and a given flow rate through the blood input line and the blood output line and (ii) identifying an offset between a measured flow rate by the first flow sensor and the second flow sensor during the known ultrafiltration rate and the given flow rate.

14. The method of claim 13, wherein establishing the second known ultrafiltration rate has a duration between 1 second and 360 seconds.

15. The method of claim 12, wherein the first flow rate in the extracorporeal circuit measured by each of the first flow sensor and the second flow sensor at a first pump flow rate and the first known ultrafiltration rate includes averaging the first measurement of the given blood flow rate in the blood input line by the first flow sensor and the second measurement of the given blood flow rate in the blood output line over a sufficient period of registered flow to include at least one full cycle of pulsatile flow signal within the average.

16. The method of claim 12, wherein the known ultrafiltration rate is a zero ultrafiltration rate.

17. A method of operating a renal replacement therapy device in an extracorporeal circuit having a pump, the renal replacement therapy device configured to provide a known ultrafiltration rate and the target ultrafiltration rate, and having a blood input line delivering blood to the renal replacement therapy device, a permeable membrane, and a blood output line passing blood from the renal replacement therapy device, the method comprising:
(a) establishing the known ultrafiltration rate and a given blood flow rate through the blood input line and the blood output line;
(b) identifying an offset between a first measurement of the given blood flow rate in the blood input line by a first flow sensor and a second measurement of the given blood flow rate in the blood output line; and
(c) adjusting, by the offset, a subsequent flow measurement of at least one of the first flow sensor and the second flow sensor.

18. The method of claim 17, further comprising (a) matching a first flow sensor configured to sense a flow rate in the blood input line and a second flow sensor configured to sense a flow rate in the blood output line, wherein the matching corresponds to at least (i) a first flow rate in the extracorporeal circuit measured by each of the first flow sensor and the second flow sensor at a first pump flow rate and a first known ultrafiltration rate, and (ii) a second flow rate in the extracorporeal circuit measured by each of the first flow sensor and the second flow sensor at a second pump flow rate and a second known ultrafiltration rate.

19. The method of claim 17, further comprising averaging the first measurement of the given blood flow rate in the blood input line by the first flow sensor and the second measurement of the given blood flow rate in the blood output line over sufficient period of registered flow to include at least one full cycle of pulsatile flow signal within the average.

20. The method of claim 17, wherein the known ultrafiltration rate is a zero ultrafiltration rate.

21. The method of claim 17, wherein establishing the known ultrafiltration rate has a duration between 1 second and 360 seconds.
